(19) United States
(12) Patent Application Publication
(10) Pub. No.: US 2012/0123540 A1
(43) Pub. Date: May 17, 2012

(54) Canine ligament bone anchor and method

(76) Inventor: John Agostini, (US)

(21) Appl. No.: 12/927,529

(22) Filed: Nov. 16, 2010

(51) Int. Cl. A61F 2/08

(52) U.S. Cl. 623/13.14

(57) ABSTRACT

The canine ligament bone anchor of the invention includes a first femoral tunnel, a transverse femoral tunnel in communication with the first femoral tunnel, the first femoral tunnel and the transverse femoral tunnel forming a chamber, an end portion of a graft positioned through the first femoral tunnel and into an inner portion of the transverse femoral tunnel, and a set bone cement disposed in the chamber.

### BACKGROUND OF THE INVENTION

The present invention relates generally to veterinary surgical procedures and more particularly to a canine ligament bone anchor and method.

Tearing or rupture of the cranial cruciate ligament (CCL) is a leading cause of lameness in a dog. Without repair of the CCL, the stifle joint will be unstable and painful and osteoarthritic degeneration can occur within months.

Known canine surgical methods of repairing the CCL include the “over and under” technique wherein a first tunnel is drilled through the distal femur entering at the origin of the CCL and exiting caudal to the lateral condyle. A second tunnel is drilled through the proximal tibia entering at the insertion of the CCL and exiting at the proximal tibial epiphysis medial to the proximal tibial tubercle. Allograft or autograft tissue is then pulled through the first and second tunnels to replace the CCL. Free ends of the tissue are sutured or screwed into fixation after proper tensioning. For example, absorbable interference screws may be used to compress the graft tissue to the walls of the first and second tunnels. Disadvantageously, failure can occur due to inherent weakening of the graft tissue during the vascularization process, poor vascularization, post-operation infection, bone fatigue, and failure of the interference screws to maintain fixation of the graft tissue. More particularly, due to its elevated tarsus, dogs constantly strain their CCL with every step leading to stretching and weakening of the graft tissue.

The known extracapsular suture technique involves using the lateral fabella and tunnels drilled in the proximal tibial tubercle for suture passage and tensioning. The suture stabilizes the stifle joint while medial collateral ligamentous and joint capsular hypertrophy create a stable knee. However, complications include soft tissue laxity that causes reduction in suture tension with modest to severe return to anterior drawer. Additionally, implant rejection necessitating removal of the surrogate ligament can occur. While this technique slows down and minimizes osteoarthritis, it does not stop all such development or progression and is not generally suitable for dogs weighing over 50 pounds.

A tibial plateau leveling technique is disclosed in U.S. Pat. No. 4,677,973 issued to Slocum. The technique includes cutting free a portion of the metaphysis of the tibia from a remaining portion of the tibia with a cylindrical cut which has a curvature axis that is perpendicular to the sagittal plane. The thus-cut proximal portion is rotated relative to the lower portion of the tibia to establish a new desired angular relation therebetween, and the two portions are then fixed relative to one another in any suitable manner, as by pinning or through the use of a bone plate. This technique provides redirection of anterior thrust forces and minimizes the effects of a missing CCL. However, complications with this technique include bone plate/screw infection necessitating removal via invasive surgery, the need for prolonged inactivity due to the osteotomy, and the need for healing in the proper alignment. Plate failure is a major complication if such failure occurs during healing as revision surgery then becomes necessary. Secondary complications include patellar ligament tendonitis. No study yet published has shown that the tibial plateau leveling technique is superior to the extracapsular technique described above.

Another known technique includes tibial tuberosity advancement in which forces acting on the tibial plateau are neutralized with a tibial tuberosity osteotomy effecting an anterior translation of the tibial insertion of the patellar ligament. The tuberosity fragment is fixed in position with a special plate and maintained at a right distance with a titanium cage. This technique is too new to be clinically proven superior to other known techniques.

It is conventional to use metal screws as bone anchoring devices to bond synthetic suture to bone. However, such metal screws cause either bone fatigue and softening, or suture erosion leading ultimately to failure of the CCL repair.

Beyar et al. disclose in U.S. Patent Application Publication No. 2007/0276392 a method of fixating soft tissue to bone including forming a tunnel in the bone, forming a radial expansion region in an inner segment of the tunnel, inserting the soft tissue into the expansion region via the tunnel and employing a fixation material capable of setting to fixate the soft tissue in the expansion region of the tunnel. The disclosed method suffers from the disadvantage that air and blood clots within the expansion region can not be visualized intra-operatively resulting in a high failure rate.

Lambert discloses in U.S. Patent Application Publication No. 2008/0027443 A1 a method for forming a bone cement anchor. The method includes disposing a graft in a bore having a major opening, a minor opening, and a tapered sidewall disposed therebetween, and then disposing a bone cement precursor in the bore such that the graft is embedded in the bone cement precursor. As applied to the repair of the anterior cruciate ligament (ACL), the method includes forming a conical or tapered bore in both the distal femur and proximal tibia, disposing a graft in the knee joint and disposing a bone cement precursor in the bores while positioning and tensioning the graft, and wherein the bone cement precursor sets to form a femoral bone cement plug and a tibial bone cement plug. Disadvantageously, the method removes large portions of cortical bone.

There remains a need in the art for a canine ligament bone anchor and method that overcomes the limitations of the prior art canine ligament bone anchor techniques.

### SUMMARY OF THE INVENTION

The canine ligament bone anchor in accordance with the invention provides a strong means for bone fixation of a synthetic graft within the distal femur. The bone anchor of the invention includes a chamber comprised of a first femoral tunnel formed in the medial aspect of the lateral femoral condyle at the origin of the CCL and a transverse femoral tunnel in communication with the first femoral tunnel. The transverse femoral tunnel extends from the lateral condyle to the medial cortex and has an axis aligned at between 65 and 90 degrees to the axis of the first femoral tunnel. A proximal opening of the first femoral tunnel is in communication with the transverse femoral tunnel so as to provide communication between the first femoral tunnel and the transverse femoral tunnel and a desired angular relationship therebetween. The chamber has a generally “T” shaped volume when the desired angle is 90 degrees and a progressively slanted “T” shaped volume as the desired angle goes from 90 degrees to 65 degrees. An end portion of a synthetic graft is positioned past the proximal opening of the first femoral tunnel into an inner portion of the transverse femoral tunnel and fixated in place by injecting a bone cement into the chamber. The resulting bone anchor formed in the chamber provides resistance to forces applied to the synthetic graft post-operatively.

The canine ligament bone anchor method in accordance with the invention provides a simple and effective technique for bone fixation of a synthetic graft within the distal femur. The method includes the steps of forming a first femoral tunnel in the distal femur, forming a transverse femoral tunnel in the distal femur, the transverse femoral tunnel being in communication with the first femoral tunnel, positioning an end portion of a graft through the first femoral tunnel and into an interior portion of the transverse femoral tunnel, and injecting bone cement into a chamber formed by the communicating first femoral tunnel and transverse femoral tunnel, the set bone cement forming a bone cement anchor into which is cemented a portion of the graft.

In one aspect of the invention, the first femoral tunnel has a diameter between 5 and 8 mm and a length of between 1.5 and 2 cm.

In another aspect of the invention, the transverse femoral tunnel has a diameter between 5 and 8 mm and a length of between 2 and 2.5 cm.

In another aspect of the invention, the end portion of the synthetic graft positioned in the inner portion of the transverse femoral tunnel substantially extends into the inner portion of the transverse femoral tunnel.

In another aspect of the invention, a transverse bone anchor portion of the bone anchor provides resistance to movement of a depending bone anchor portion to prevent the depending bone anchor portion from moving within the first femoral tunnel.

In another aspect of the invention, the first femoral tunnel and the transverse femoral tunnel have cylindrical walls.

In another aspect of the invention, the first femoral tunnel formed in the distal femur comprises a distal opening formed at the medial aspect of the lateral femoral condyle at the origin point of the cranial cruciate ligament and a proximal opening in communication with the inner portion of the transverse femoral tunnel.

In another aspect of the invention, the transverse femoral tunnel formed in the distal femur comprises a lateral opening formed at the lateral condyle and a medial wall formed in the medial cortex, the first femoral tunnel proximal opening being in communication with the inner portion of the transverse femoral tunnel at a position substantially midway between the lateral opening and the medial wall.

There has been outlined, rather broadly, the more important features of the invention in order that the detailed description thereof that follows may be better understood, and in order that the present contribution to the art may be better appreciated. There are, of course, additional features of the invention that will be described below and which will form the subject matter of the claims appended herein.

In this respect, before explaining at least one embodiment of the invention in detail, it is to be understood that the invention is not limited in its application to the details of design and to the arrangement of the components set forth in the following description or illustrated in the drawings. The invention is capable of other embodiments and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other methods and systems for carrying out the several purposes of the present invention. It is important, therefore, that the claims be regarded as including such equivalent methods and systems insofar as they do not depart from the spirit and scope of the present invention.

### BRIEF DESCRIPTION OF THE DRAWINGS

The present disclosure may be better understood and its numerous features and advantages made apparent to those skilled in the art by referencing the accompanying drawings wherein:

FIG. 1 is a schematic illustration of a right knee joint in flexion wherein a first femoral tunnel and a tibial tunnel have been formed;

FIG. 2 is a schematic illustration of the right knee joint of FIG. 1 wherein a transverse femoral tunnel has been formed in the distal femur in communication with the first femoral tunnel;

FIG. 3 is a schematic illustration of the right knee joint of FIG. 2 wherein a graft has been positioned within a chamber formed by the first femoral tunnel and the transverse femoral tunnel; and

FIG. 4 is a schematic illustration of the right knee joint of FIG. 3 wherein a set bone cement disposed within the chamber anchors the positioned graft to the distal femur.

### DETAILED DESCRIPTION OF THE INVENTION

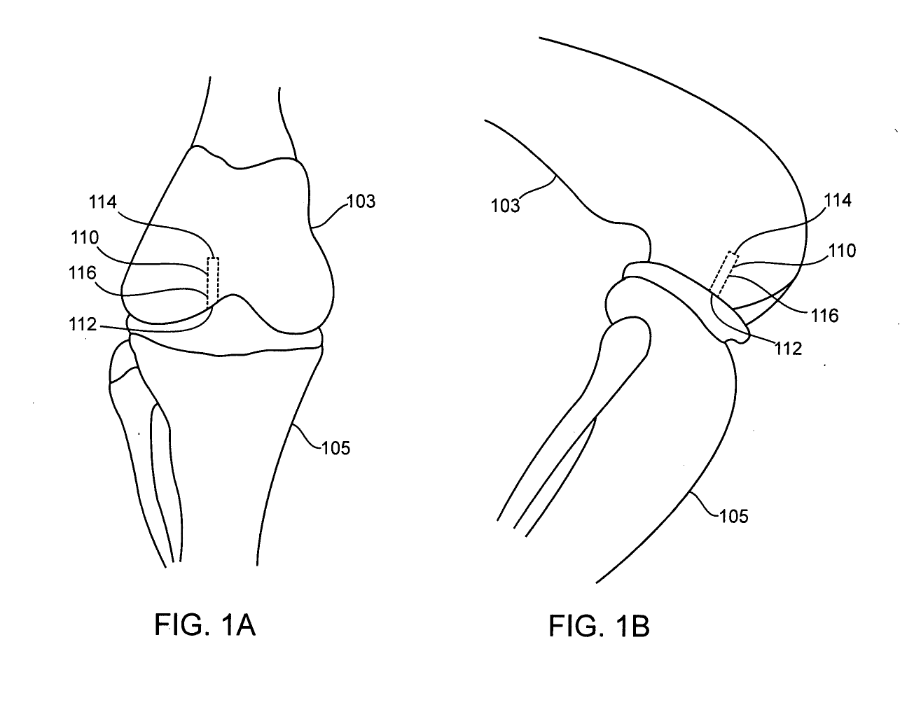

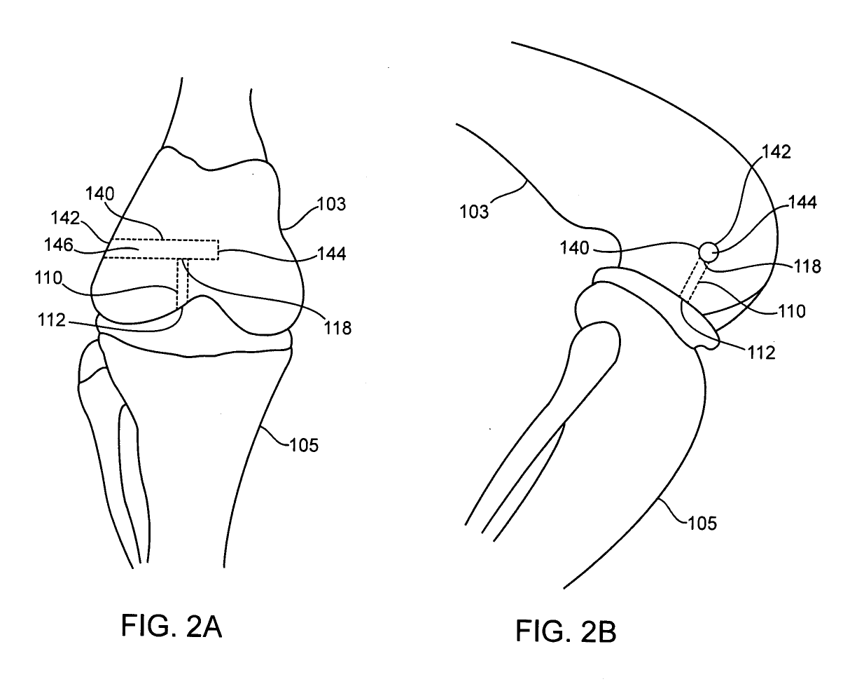

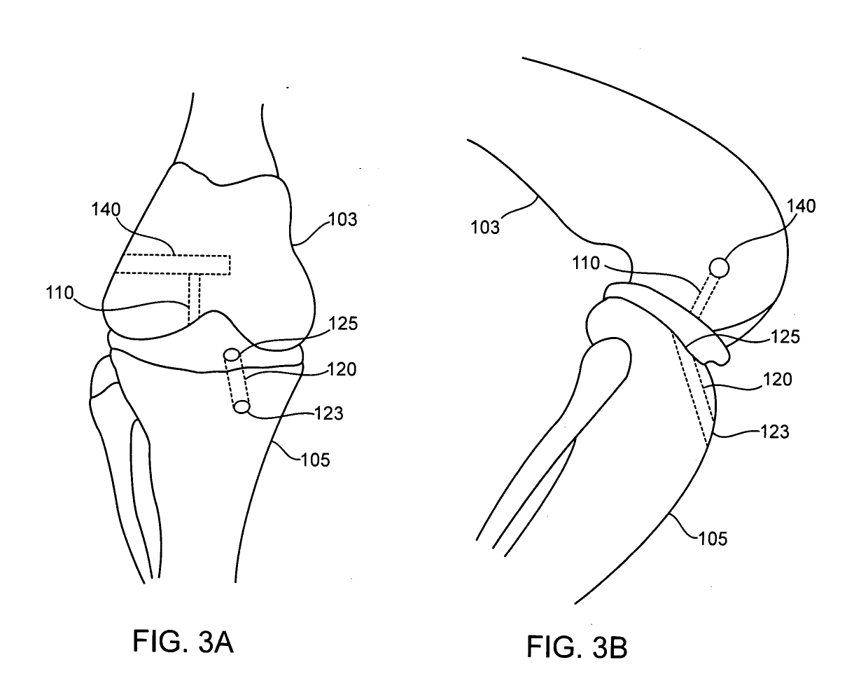

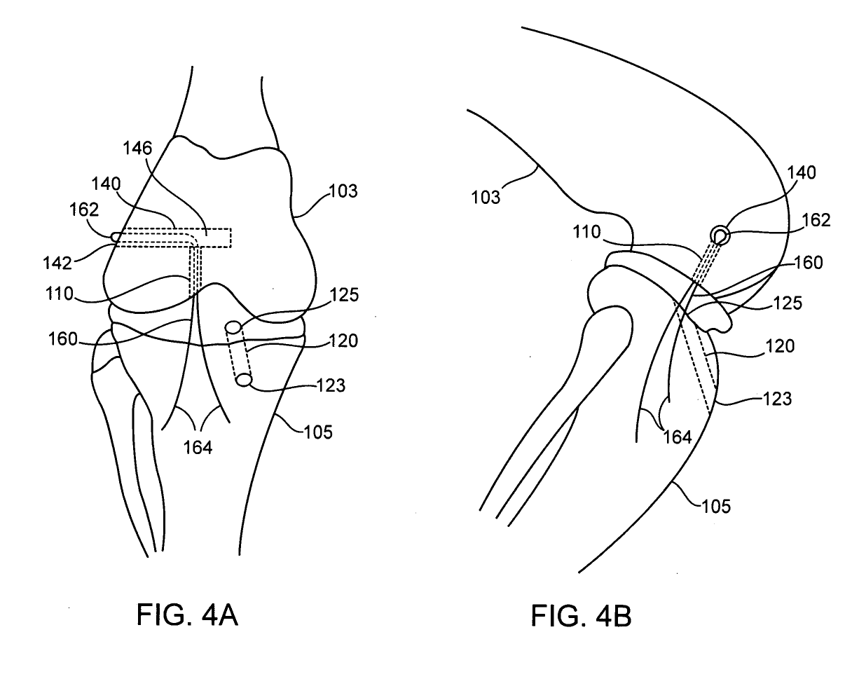

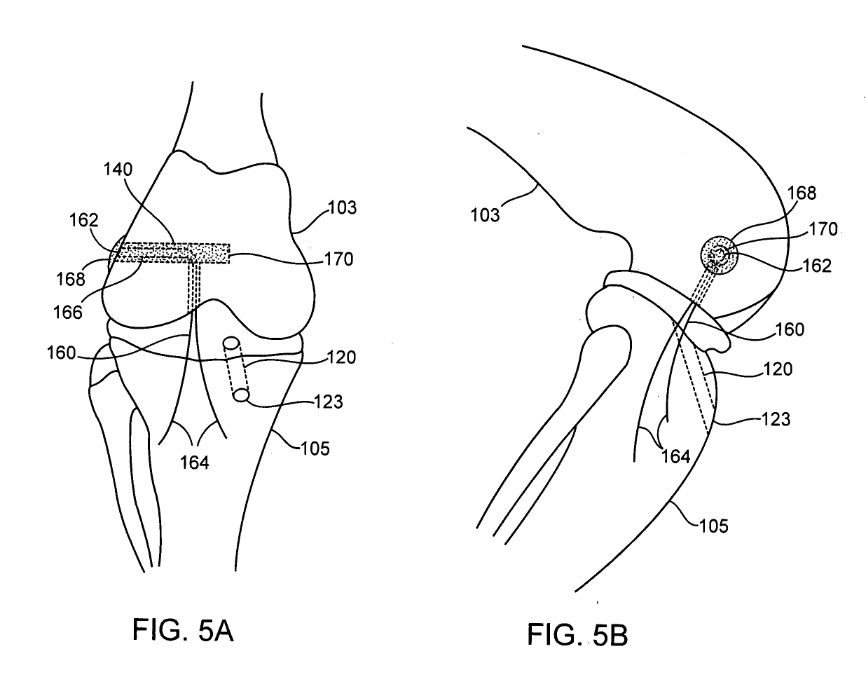

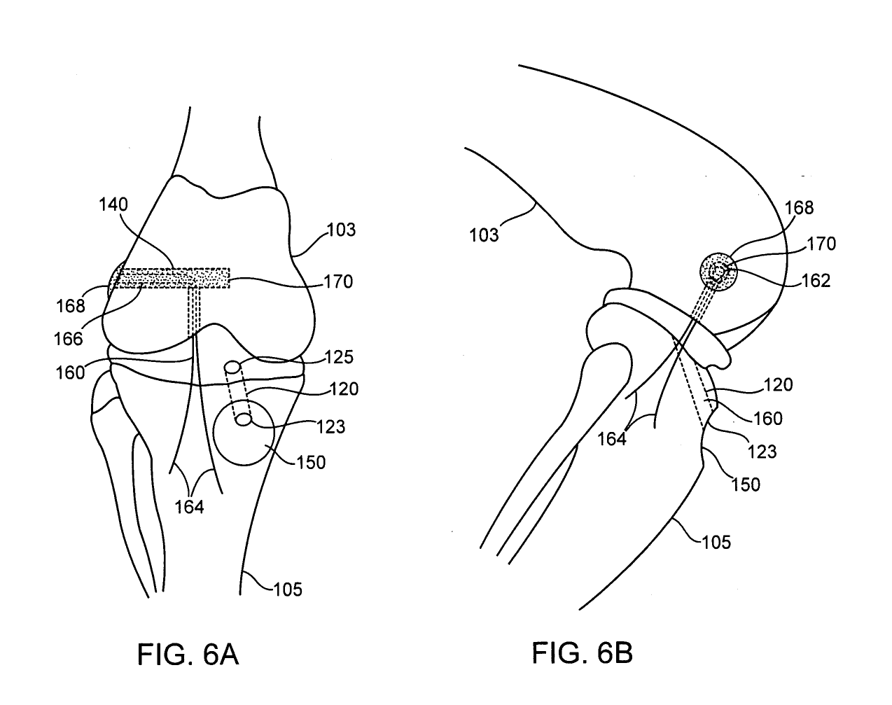

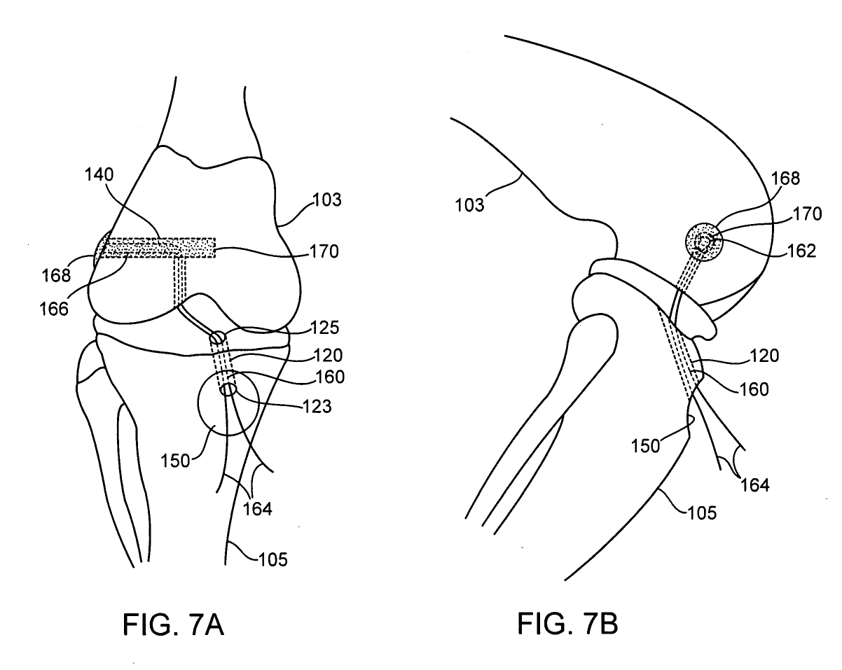

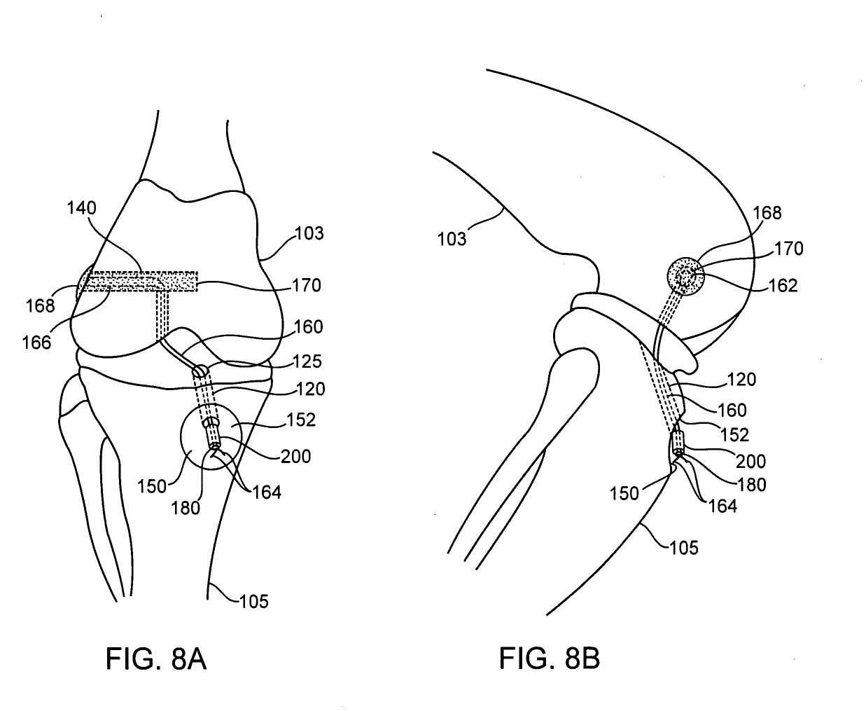

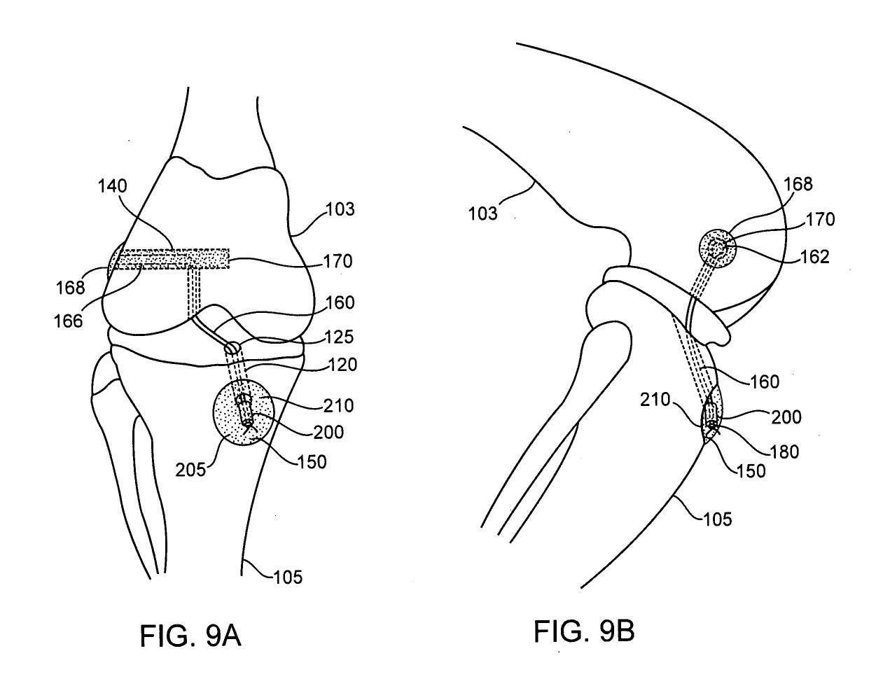

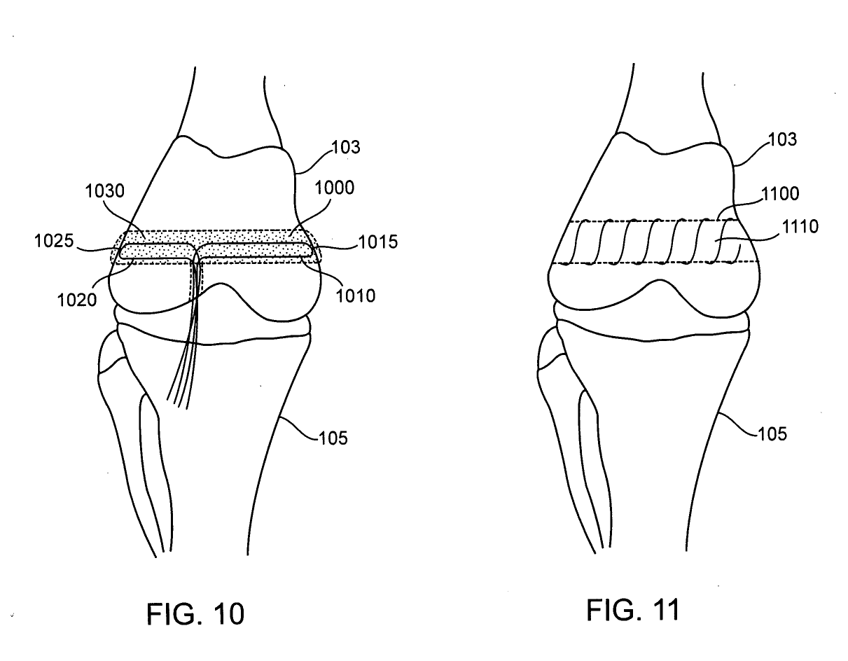

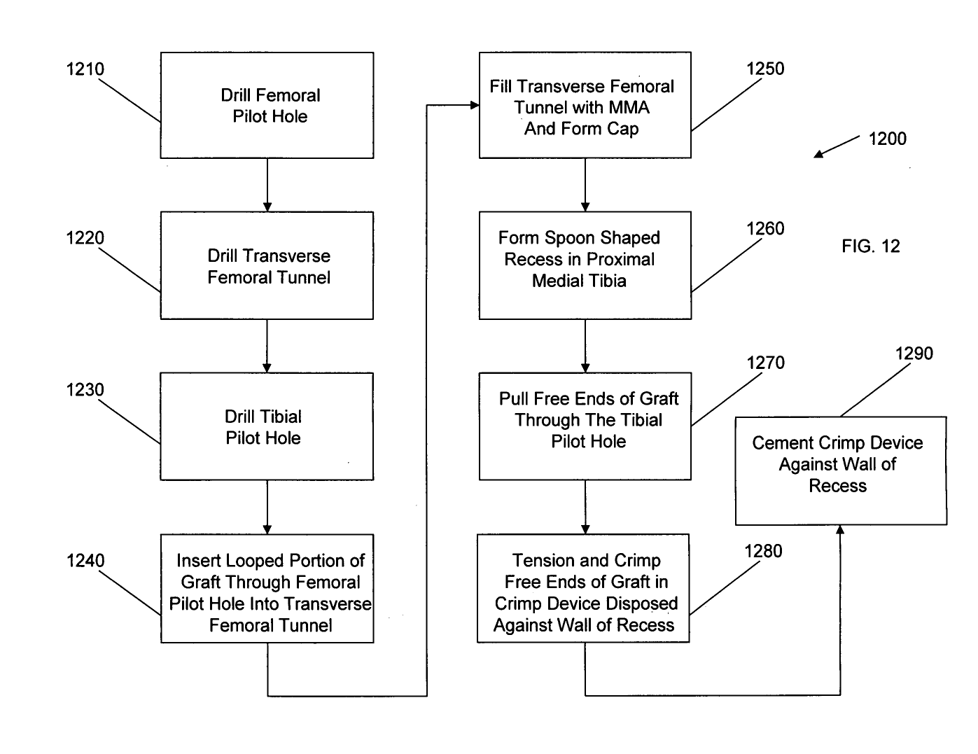

The invention will now be described with reference to FIGS. 1-4. A canine right knee including a femur 100 and a tibia 105 is schematically shown in each figure in flexion. A canine ligament bone anchor method in accordance with the invention includes a step in which a first femoral tunnel 110 is formed in the distal femur. The first femoral tunnel 110 is formed in the medial aspect of the lateral femoral condyle at the origin of the CCL based on considerations well known in the art. The first femoral tunnel 110 has a diameter of between 5 and 8 mm and a length of between 1.5 and 2 cm. A distal opening 112 of the first femoral tunnel 110 communicates with the intercondylar notch and a proximal wall 114 is disposed in the medial cortex. The first femoral tunnel is preferably of cylindrical configuration and has an axis A-A and a wall 116 extending from the distal opening 112 to the proximal wall 114.

In a next step a transverse femoral tunnel 140 is formed in the distal femur. The transverse femoral tunnel 140 extends from a lateral opening 142 at the lateral condyle to a medial wall 144 at the medial cortex and has an axis B-B aligned at an angle α between 65 and 90 degrees to the axis A-A of the first femoral tunnel. The transverse femoral tunnel 140 has a diameter of between 5 and 8 mm and a length of between 1.5 and 4.5 cm. The transverse femoral tunnel 140 is formed such that the proximal wall 114 of the first femoral tunnel 110 is opened into an inner portion 146 of the transverse femoral tunnel 140 at a proximal opening 118 of the first femoral tunnel 110. The proximal opening 118 is preferably disposed substantially midway between the lateral opening 142 and the medial wall 144 of the transverse femoral tunnel 140. The intersection of, and communication between, the transverse femoral tunnel 140 and the first femoral tunnel 110 provides a chamber 150. The chamber 150 has a generally “T” shaped volume when the angle α is 90 degrees and a progressively slanted “T” shaped volume as the angle α goes from 90 degrees to 65 degrees.

In a next step, an end portion 162 of a graft 160 is positioned through the first femoral tunnel 110 and into the inner portion 146 of the transverse femoral tunnel 140. In a preferred embodiment the graft 160 is looped nylon cord and the end portion 162 is a loop. The end portion 162 of the graft 160 may extend substantially into the inner portion 146 of the transverse femoral tunnel 140.

In a last step, bone cement 170 is injected into the chamber 150 formed by the communicating first femoral tunnel 110 and transverse femoral tunnel 140, the set bone cement forming a bone cement anchor 180 into which is embedded a portion 164 of the graft 160 including the end portion 162. The bone cement is preferably polymethyl methacrylate. The bone cement anchor 180 formed in the chamber 150 provides resistance to forces applied to the graft 160 post-operatively. A transverse portion 182 of the bone cement anchor 180 provides resistance to movement of a depending bone anchor portion 184 to prevent the depending bone anchor portion 184 from moving within and/or out of the first femoral tunnel 110.

The bone cement anchor 180 provides a means of permanently and securely attaching the graft 160 to the distal femur such that the portion 164 of the graft 160 is firmly embedded in the bone cement anchor 180. The graft 160 thus attached may then be positioned through a tibial tunnel 120 and tensioned using known techniques including tying a knot 190 and using a crimp 195.

The canine ligament bone anchor method in accordance with the invention provides a surgical technique wherein the cylindrical configuration of the first femoral tunnel and of the transverse femoral tunnel provides the surgeon with full visualization and control over air pockets and blood clots during the liquid to solid set points of the bone cement. Additionally, the canine ligament bone anchor can be replaced if failure occurs operatively or post operatively without additional trauma to the tunneled bones of the patient.

The foregoing description of the embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

I claim:

1. A method of forming a canine ligament bone anchor comprising the steps of:
forming a first femoral tunnel in the distal femur;
forming a transverse femoral tunnel in the distal femur, the transverse femoral tunnel being in communication with the first femoral tunnel;
positioning an end portion of a graft through the first femoral tunnel and into an interior portion of the transverse femoral tunnel; and
injecting bone cement into a chamber formed by the communicating first femoral tunnel and transverse femoral tunnel, the set bone cement forming the ligament bone anchor into which is cemented a portion of the graft.

2. The method of claim 1, wherein the first femoral tunnel formed in the distal femur comprises a distal opening formed at the medial aspect of the lateral femoral condyle at the origin point of the cranial cruciate ligament and a proximal opening in communication with the inner portion of the transverse femoral tunnel.

3. The method of claim 2, wherein the transverse femoral tunnel formed in the distal femur comprises a lateral opening formed at the lateral condyle and a medial wall formed in the medial cortex, the first femoral tunnel proximal opening being in communication with the inner portion of the transverse femoral tunnel at a position substantially midway between the lateral opening and the medial wall.

4. A canine ligament bone anchor comprising:
a first femoral tunnel;
a transverse femoral tunnel in communication with the first femoral tunnel, the first femoral tunnel and the transverse femoral tunnel forming a chamber;
an end portion of a graft positioned through the first femoral tunnel and into an inner portion of the transverse femoral tunnel; and
a set bone cement disposed in the chamber.

5. The canine ligament bone anchor of claim 4, wherein the transverse femoral tunnel has an axis aligned with an axis of the first femoral tunnel at an angle between 65 and 90 degrees.

6. The canine ligament bone anchor of claim 4, wherein the chamber has a “T” shaped volume in the case where the axes are aligned at 90 degrees.

7. The canine ligament bone anchor of claim 4, wherein the first femoral tunnel comprises a proximal opening in communication with the inner portion of the transverse femoral tunnel and the proximal opening is disposed substantially midway between a lateral opening and a medial wall of the transverse femoral tunnel.

8. The canine ligament bone anchor of claim 4, wherein the graft is a looped nylon cord and the end portion is a loop.

9. The canine ligament bone anchor of claim 4, wherein the set bone cement comprises polymethyl methacrylate.

10. The canine ligament bone anchor of claim 4, further comprising a transverse portion and a portion depending from the transverse portion, the transverse portion providing resistance to movement of the depending portion.

11. The canine ligament bone anchor of claim 4, wherein the first femoral tunnel has a diameter of between 5 and 8 mm and a length of between 1.5 and 2 cm.

12. The canine ligament bone anchor of claim 4, wherein the transverse femoral tunnel has a diameter of between 5 and 8 mm and a length of between 1.5 and 4.5 cm.

13. The canine ligament bone anchor of claim 4, wherein the first femoral tunnel and the transverse femoral tunnel have cylindrical walls.
